(19) United States
(12) Patent Application Publication
(10) Pub. No.: US 2015/0090057 A1
(43) Pub. Date: Apr. 2, 2015

(54) COMPONENTS AND METHODS FOR A CATHETER POSITIONING SYSTEM WITH REMOVABLE CATHETER ACTUATOR COLLARS

(71) Applicant: Catheter Robotics Inc., Budd Lake, NJ (US)

(72) Inventors: Robert Pacheco, Bayside, NY (US); Steve Foley, Kerrville, TX (US); David Jenkins, Budd Lake, NJ (US); Thomas Jackson, Cambridge (GB); Luke Hares, Milton (GB)

(21) Appl. No.: 14/494,709

(22) Filed: Sep. 24, 2014

Related U.S. Application Data
(60) Provisional application No. 61/883,323, filed on Sep. 27, 2013.

(51) Int. Cl. A61M 25/01; F16H 19/02

(52) U.S. Cl. CPC A61M 25/0105; F16H 19/02
USPC 74/25

(57) ABSTRACT

Embodiments include removable catheter actuator collars which may connect a catheter actuator on a catheter's handle to a catheter positioning device. The removable catheter actuator collars may allow the catheter to be easily removed from the catheter positioning device by a physician. The removable catheter actuator collars may remain connected with the catheter when removed and may allow the catheter to be easily reconnected to the catheter positioning system. In further embodiments, the removable catheter actuator collars may be annular or ring shaped and secure to a catheter actuator with various mechanisms such as one or more clamps, compression grips, or rotating portions and may remain connected with the catheter when the catheter is removed from the catheter positioning system.

### CROSS-REFERENCE TO RELATED APPLICATIONS

This application claims the benefit of priority to U.S. Provisional Patent Application No. 61/883,323, entitled “COMPONENTS AND METHODS FOR A CATHETER POSITIONING SYSTEM WITH REMOVABLE CATHETER ACTUATOR COLLARS,” filed Sep. 27, 2013, the entire contents of which are incorporated herein by reference.

### BACKGROUND

Many procedures involving catheter insertion, such as invasive electrophysiology procedures, rely on fluoroscopy or other radioactive imaging techniques to help navigate and position the catheter within a patient's body at a particular site, such as in the heart or inside a blood vessel in the circulatory system. High dosages of radiation can have long term adverse health effects. A patient may be directly exposed only once or twice to radiation during such procedures and avoid such adverse effects. However, physicians, medical technicians and staff can experience a large cumulative radiation dosage over time, both directly and indirectly, from conducting many procedures.

To protect the operator and staff from this radiation, shielding such as lead aprons, gowns, glasses, skirts, etc., is worn. Such lead clothing, especially a lead apron, is quite heavy and uncomfortable, and its use has been associated with cervical and lumbar spine injury or degradation.

### SUMMARY OF THE INVENTION

Various embodiments provide a catheter positioning device that may integrate with a catheter via one or more removable catheter actuator collars that allow the catheter to be removed from the catheter positioning device for manual positioning In various embodiments, the removable catheter actuator collars may secure to a catheter actuator with various mechanisms such as one or more clamps, compression grips, or latches and may be removed with the catheter from the catheter positioning system. In further embodiments, the removable catheter actuator collars may couple with the catheter positioning device via one or more projections or gear teeth.

### BRIEF DESCRIPTION OF THE DRAWINGS

The accompanying drawings, which are incorporated herein and constitute part of this specification, illustrate exemplary embodiments of the invention, and together with the general description given above and the detailed description given below, serve to explain the features of the invention.

FIG. 1 is a system block diagram illustrating a remote controller, a remotely controlled catheter system, and a programmable control system.

FIG. 2A is an oblique view of an embodiment removable catheter actuator collar with a compression grip.

FIG. 2B is an oblique view of the removable catheter actuator collar of FIG. 2A attached to a rotating catheter actuator of a catheter handle.

FIG. 2C is an oblique view of the removable catheter actuator collar of FIG. 2A and FIG. 2B coupled to a sled member of a catheter positioning system.

FIG. 3A is an oblique view of an embodiment removable catheter actuator collar with a clasp.

FIG. 3B is an oblique view of the removable catheter actuator collar of FIG. 3A coupled to a sled member of a catheter positioning system.

FIG. 4A is an oblique view of an embodiment removable catheter actuator collar with a latch.

FIG. 4B is an oblique view of an embodiment removable catheter actuator collar having a projection and coupled to a sled member of a catheter positioning system.

FIG. 5A is an oblique view of an embodiment removable catheter actuator collar coupled with a modular plate.

FIG. 5B is a cross sectional profile view of the removable catheter actuator collar of FIG. 5A and ridges disposed in a clasp of a modular plate.

FIG. 6A is an oblique view of an embodiment removable catheter actuator collar in an open position.

FIG. 6B is an oblique view of the embodiment removable catheter actuator collar of FIG. 6A with a portion rotated to a closed position.

FIG. 6C is an oblique view of the embodiment removable catheter actuator collar of FIG. 6A coupled with a catheter actuator.

FIG. 7A is an oblique view of an embodiment removable catheter actuator collar in an open position.

FIG. 7B is an oblique view of the embodiment removable catheter actuator collar of FIG. 7A with a portion rotated to a closed position.

FIG. 7C is an oblique view of the embodiment removable catheter actuator collar of FIG. 7A coupled with a catheter actuator.

FIG. 8 is oblique view of the removable catheter actuator collar coupled to a sled member of a catheter positioning system.

FIG. 9 is an oblique view of a portion of an embodiment removable catheter actuator collar with a snap.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the invention or the claims.

Various embodiments provide improved removable catheter actuator collars enabling control of a catheter with a catheter positioning device. The catheter positioning device enables a physician to remotely control manipulation and insertion of a catheter into a patient while being positioned away from sources of radiation used for imaging or other procedures. The catheter positioning device may be used to move the attached catheter, such as rotating, advancing, or retracting the catheter in relation to a patient or within a patient's body. An example of a catheter positioning device is disclosed in PCT Application PCT/US2009/031357, which published as WO 2009/092059 and is incorporated herein by reference in its entirety.

The catheter positioning device may also be used to actuate the catheter, such as by controlling an actuator on a catheter handle. Catheter actuators may perform various tasks, such as deflecting a tip to help in navigation or controlling one or more transducers to assist in an operation. In various embodiments, one or more removable catheter actuator collars may be used to connect one or more catheter actuators to the catheter positioning device in order to remotely control the catheter actuators.

During an operation, a physician may wish to manually control the catheter rather than remotely controlling the catheter with the catheter positioning system. The removable catheter actuator collars may enable the catheter to be easily removed from the catheter positioning device by the physician or operator. The removable catheter actuator collars may remain connected with the catheter when the catheter is removed and may enable the catheter to be easily reconnected to the catheter positioning system by the physician or operator.

In various embodiments, the removable catheter actuator collars may be annular or ring shaped and may be secured to a catheter actuator with various mechanisms such as one or more clamps, compression grips, or rotating portions and may be removed along with the catheter from the catheter positioning system. In further embodiments, the removable catheter actuator collars may couple, engage or interface with the catheter positioning device with one or more projections or gear teeth. In the various embodiments, the removable catheter actuator collars may be sterile components, either sterilizable or disposable, to avoid introducing contaminants into the body of a patient. For example, removable catheter actuator collars that interface with the catheter may be sterile and disposable, or sterilizable.

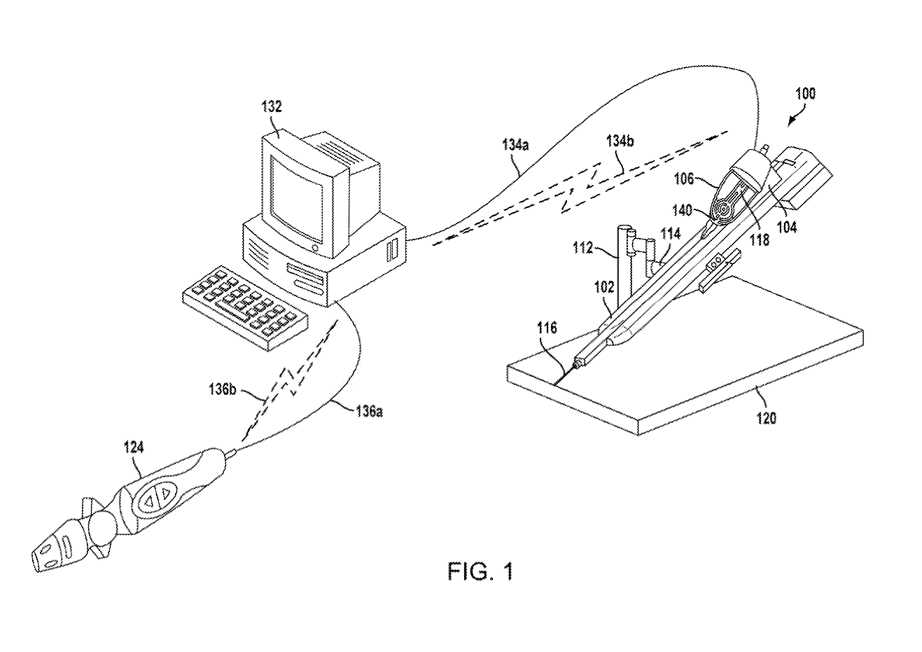

FIG. 1 illustrates an embodiment catheter positioning device 100 with a remote controller 124. The catheter positioning device 100 may include a sled base 102 coupled with a sled member 104. The sled base 102 may be configured to advance or retract the sled member 104 along the sled base 102 towards the body of the patient or back away from the patient. For example, the sled member may be moved with a motor at one end of the sled base 102. The sled member 104 may be driven back and forth along a rail or sled base 102 by a drive mechanism, such as a worm drive, in order to advance or withdraw the catheter.

The sled base may be held in position above a patient or operating table 120 by a bridge (not shown) or support arm 112 that includes a sled base support structure 114 that holds the sled base 102 in a fixed position and orientation. The arm 112 may be configured with articulating joints such that the arm 112 may be extended or rotated to position the sled base 102 relative to a patient on the operating table 120. The sled base may also include a nose cone 116 that supports insertion of the catheter into a patient. A catheter may be advanced along the sled base 102 by the sled member 104 so that it passes through the nose cone 116 and into the patient.

The sled base 102 may include a sterile barrier configured to support and protect the catheter. The sterile barrier may include a resealable delivery channel configured to receive and guide the catheter along the sled base as it is advanced or retracted by the sled member 104. For example, the catheter may be inserted into the delivery channel and then the catheter handle 118 may be connected to the sled member 104 (such as by using the modular plate 106 discussed below) such that the catheter is driven forward or backward by translation of the sled member 104 along the resealable delivery channel in the sled base 102 and through the nose cone 116 into or out of the patient.

The sled member 104 may be equipped with a modular plate 106 to which a catheter handle 118 may be attached. Many alternate modular plates 106 may be configured to adapt to different types of catheter handles for different catheters and catheter controls. The modular plates 106 may otherwise be consistent, such as with regard to a mechanical interface by which the modular plate 106 attaches to the sled member 104. The different modular plates 106 may be swapped in and out so that the catheter positioning system may be used with many different types of catheters. Depending on the kind of catheter that is desired for a procedure, an appropriate modular plate 106 may be attached to the sled member 104 and the catheter may be attached to the module plate 106, such as by one or more removable catheter actuator collars 140. The modular plate 106 and/or removable catheter actuator collars 140 may integrate with any actuators on the catheter handle 118 thereby allowing an operator to control the actuators via the remote controller 124.

The sled member 104 may be rotated by a drive mechanism in order to rotate a catheter connected to the modular plate 106. The rotation of the sled member 104 may be controlled remotely via the remote controller 124. By controlling translation along the sled base 102, rotation of the sled member 104, and actuation of the catheter handle via the modular plate 106, an operator may position or use the catheter in any way necessary for a desired operation. Further, an operator may control each of these degrees of freedom (i.e., translation, rotation, and actuation) remotely with the remote controller 124.

A remote controller 124 may be connected to a programmable control system 132 by a wired connection 136a or a wireless connection 136b, such as a wireless data link. The programmable control system 132 may also be connected to the catheter positioning device 100 by a wired connection 134a or a wireless connection 134b such as a wireless data link.

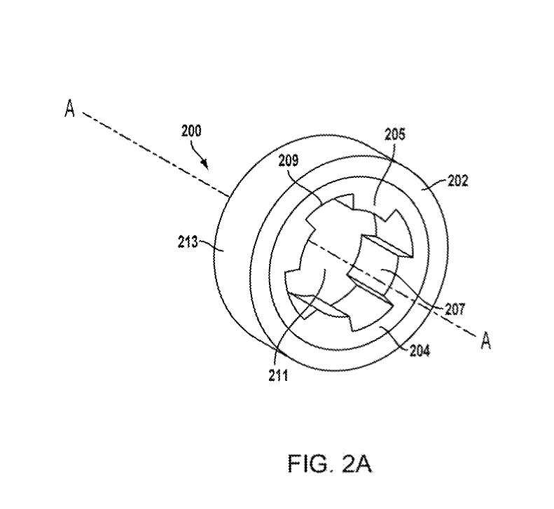

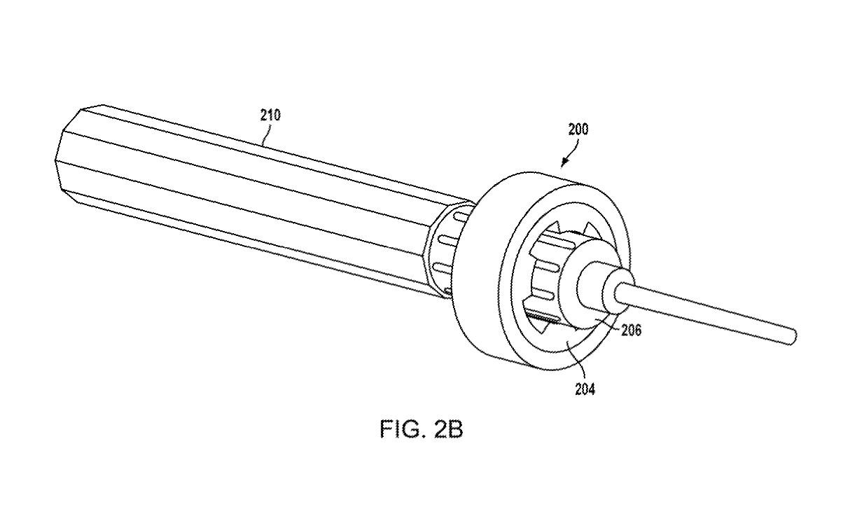

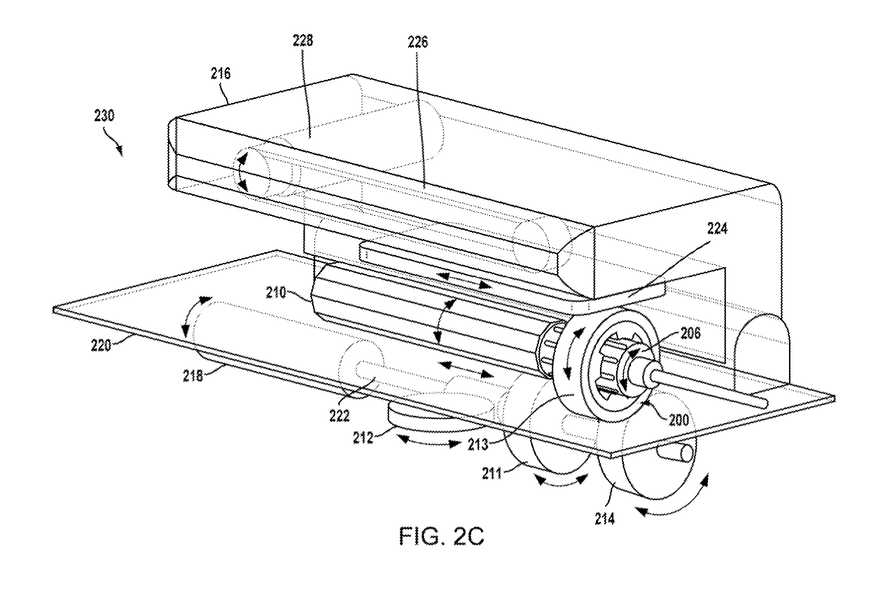

FIG. 2A and FIG. 2B illustrate an embodiment removable catheter actuator collar 200 with an annular shape that may be placed over a catheter handle 210. In embodiments, as illustrated in FIG. 2A, the removable catheter actuator collar 200 may be a two piece ring having an outer ring 202 surrounding an inner ring 204. The inner ring 204 may be centered within the central opening of the outer ring 202. The inner surface of the outer ring 202 may be coupled to the outer surface of the inner ring 204 such that outer ring 202 and inner ring 204 may rotate together about a central axis A. In an embodiment, the outer ring 202 and the inner ring 204 may be formed from different materials. As an example, the outer ring 202 may be formed of a material that is less elastic (e.g., molded plastic) than the material comprising the inner ring 204 (e.g., rubber). In an embodiment, the inner ring 204 may include one or more protrusions 205 (e.g., teeth) extending inward from the circumference 209 of the inner ring 204 into a central opening 211 of the inner ring toward the central axis A. For example, one, two, three, four, or more protrusions 205 may extend into the central opening 211. Said another way, the inner ring may include a number of channels 209 that leave protrusions 205 within the central opening 211. When deformed (e.g., compressed), the one or more protrusions 205 may be configured to exert a force in the opposite direction of the force of the deformation (e.g., compression) to resist the deformation (e.g., compression). The deformation may be resisted due to the outer ring 202, e.g., because the outer ring 202 may prevent the outer circumference of the inner ring 204 from expanding in response to the deformation forces. In this manner, as shown in FIG. 2B the catheter handle 210 may be inserted into the central opening 211 (or the opening 211 may be placed over the catheter handle 210) and the one or more protrusions 205 may be compressed and may exert a force against the catheter handle 210. The compressive and deformative forces acting on the protrusions 205 may enable the catheter actuator collar 200 to grip the catheter handle 210. In an embodiment shown in FIG. 2A, the surface 207 of the one or more protrusions 205 may be curved. In an embodiment, the outer surface 213 of the outer ring 202 may be a smooth surface. In another embodiment, the outer surface 213 of the outer ring 202 may be configured to form a gear including one or more teeth. In an embodiment, the catheter actuator collar 200 may be removed from the catheter handle 210 by sliding the catheter actuator collar 200 off the catheter handle 210.

As shown in FIG. 2B, the catheter handle 210 may include a rotating actuator 206, which may be inserted through the central opening 211 in the inner ring 204 such that the one or more protrusions 205 of the inner ring 204 may grip the rotating actuator 206. The teeth 205 may flex as the catheter handle 210 is inserted and may push against the actuator 206 forming a secure coupling such that a rotation of the actuator collar 200 may cause the actuator 206 to rotate. In this manner, rotation of the removable catheter actuator collar 200 may rotate the rotating actuator 206 and actuate the associated catheter function (e.g., catheter rotation).

The removable catheter actuator collar 200 may enable a catheter positioning system to control the catheter actuator 206 in connection with other movements in addition to rotation. FIG. 2C illustrates a sled member 230 of a catheter positioning system that may interface with the removable catheter actuator collar 200 to rotate the rotating actuator 206 of the catheter handle 210. The sled member 230 may include a first face 220 and second face 216 that are configured to enclose around the catheter handle 210, such as on opposite sides of the catheter handle 210. In alternate embodiments, the sled member 230 may have a single face or more than two faces.

Each face 220, 216 of the sled member may have one or more motors and/or drive shafts. For example, as illustrated in FIG. 2C, the first face 220 may include a motor 218 connected to an axle 222 connected to one or more rotating linkages, such as linkages 212 and 214. The axle 222 may be engaged by a bushing or linkage 212. The rotating linkage 214 may interface with outer surface 213 of the removable catheter actuator collar 200. When the motor 218 turns the axle 222 and rotating linkage 214, the removable catheter actuator collar 200 may rotate and thereby rotate the rotating actuator 206. In further embodiments, a linkage 211 may be used to rotate the catheter handle 210 independently from the rotating actuator 206.

As illustrated in FIG. 2C, the face 216 may be provided with a motor 228, which may be coupled to and drive a belt 226 or other drive mechanism. The belt 226 may be coupled to mechanism configured to move a drive plate 224 along a linear axis. The drive plate 224 may be coupled to the catheter handle 210 and/or may come into frictional contact with the outer surface 213 of the removable catheter actuator collar 200. The drive plate 224 may move the catheter handle 210 and/or the removable actuator collar 200 (and the rotating actuator 206) back and forth in a linear direction. In some embodiments, the linkage 212 may be used to adjust the position of the linkage 214 and the linkage 211 such that rotational movement of the removable actuator collar 200 (and the rotating actuator 206) and rotational movement of the catheter handle 210 may continue to be accomplished.

The rotating linkage 214 may be a permanent part of the catheter positioning system. The removable catheter actuator collar 200 may detach or decouple from rotating linkage 214 such that the removable catheter actuator collar 200 together with the catheter held by the removable catheter actuator collar 200 may be removed from the sled member 230 and the catheter positioning system.

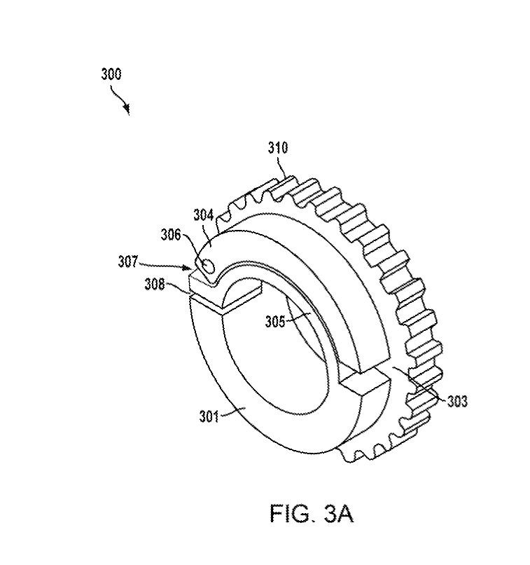

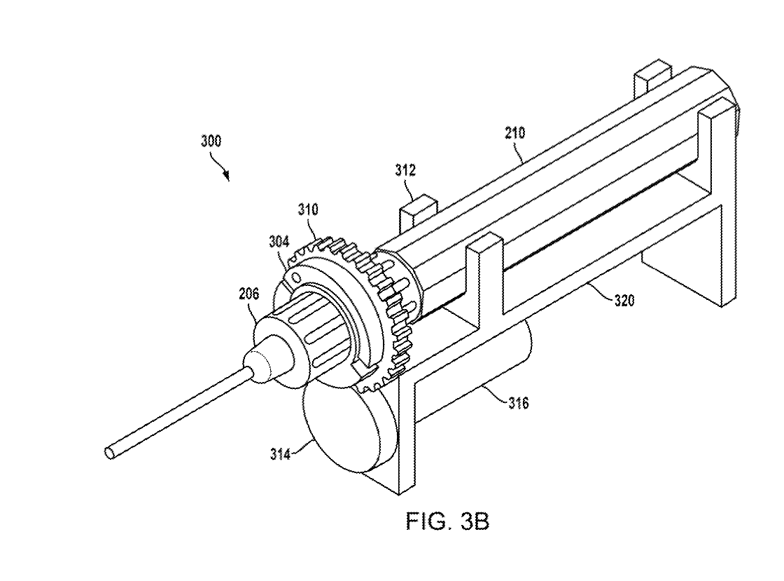

FIG. 3A illustrates an embodiment removable catheter actuator collar 300 that may be attached to a rotating actuator of a catheter handle, such as the rotating actuator 206 of the catheter handle 210 describe above. The removable catheter actuator collar 300 may comprise an annular or a ring-shaped body having main body section 303 and an adjustable clamping section 301. The main body section 303 may have an inner circumference and gear teeth 310 extending from an outer circumference. The adjustable clamping section 301 may comprise a clamping arm 305 forming a length of the inner circumference of the adjustable clamping section 301. A clamp 304 may be rotationally coupled to the main body section 303 by a pin 306 and the clamp 304 may rotate about the pin 306. In an open position, the clamp 304 may exert less force on the clamping arm 305 than in a closed position. When the clamp 304 is in an open position the inner circumference of the adjustable clamping section 301 may be larger than in a closed position. The clamp 304 may be provided with a cam portion 307. As the clamp 304 rotates about the pin 306 from the open position to the closed position, the clamping arm 305 may be deflected toward the center of the opening in the adjustable clamping section 301, such as by the action of the cam portion 307 on the clamping arm 305. Deflection of the clamping arm 305 may reduce the circumference of the adjustable clamping section 301 and reducing the size of the gap 308 in the adjustable clamping section 301 when compared with the open position. In the closed position the circumference of the adjustable clamping section 301 may be reduced such that the adjustable clamping section 301 may securely grip a rotating actuator of a catheter handle. In an embodiment, the catheter actuator collar 300 may be removed from an actuator of a catheter handle by rotating the clamp 304 to an open position, thereby increasing the circumference of the adjustable clamping section 301 enabling the catheter actuator to be slid out of the catheter actuator collar 300.

FIG. 3B illustrates the removable catheter actuator collar 300 affixed around the rotating catheter actuator 206 of the catheter handle 210. The clamp 304 is illustrated in a closed position, which may cause the clamping arm 305 of the adjustable clamping section to securely grip the rotating catheter actuator 206. The catheter handle 210 may be coupled with a sled member 320 by one or more clamps, retaining structures, frame structures, etc., such as clasps 312. The sled member 320 may have a motor 316 that drives a rotating linkage 314. The geared teeth 310 of the removable catheter actuator collar 300 may interface with corresponding gear teeth (not shown) of the rotating linkage 314. When the motor 316 turns the rotating linkage 314, the removable catheter actuator collar 300 will also rotate thereby rotating the rotating actuator 206. In some embodiments, the rotating linkage 314 may be a permanent part of the catheter positioning system. The removable catheter actuator collar 300 may detach or decouple from rotating linkage 314 such that the removable catheter actuator collar 300 together with the catheter handle 210 held by the removable catheter actuator collar 300 may be removed from the clasp 312 of the sled member 320 and the catheter positioning system. After removal, the removable catheter actuator collar 300 may be sterilized, resterilized or disposed of.

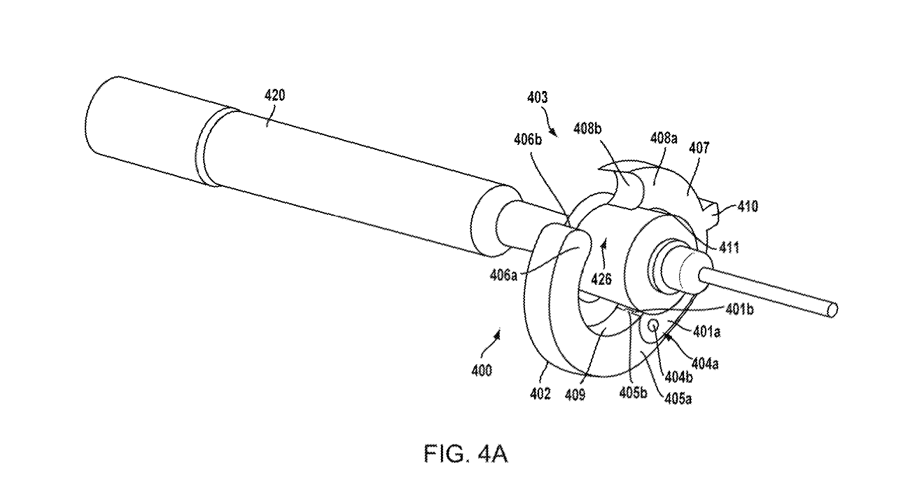

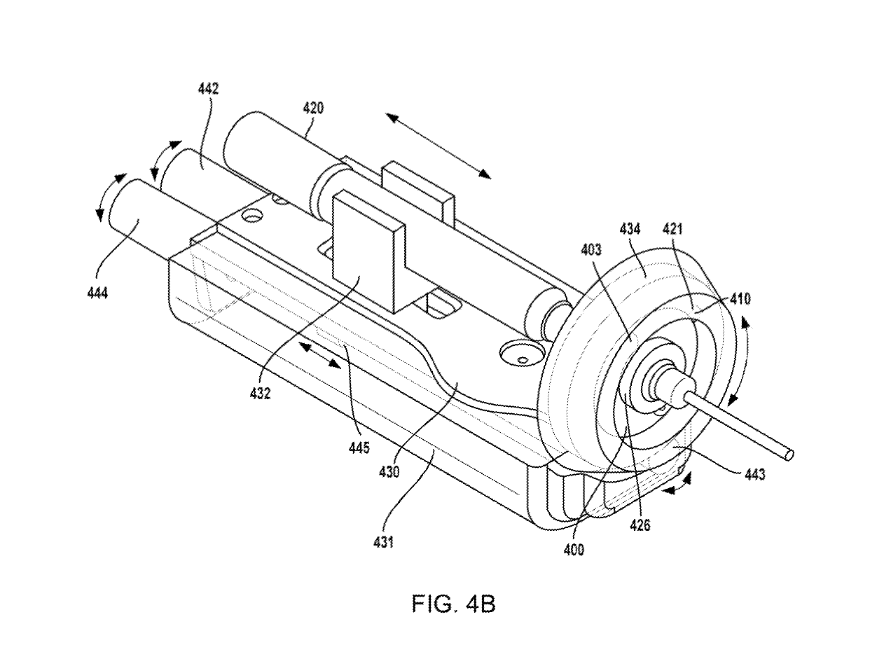

FIG. 4A illustrates another embodiment removable catheter actuator collar 400 that may fasten about a rotating actuator 426 of a catheter handle 420. The removable catheter actuator collar 400 may include a first semi-circular portion 402 and a second semi-circular portion 407. The first semi-circle portion 402 may be rotationally coupled to the second semi-circle portion 407, around a hinged joint 404a around which the first semi-circle portion 402 and second semi-circle portion 407 may rotate. For example, the hinged joint 404a may include a pin 404b that couples a first end 405a of the first semi-circle portion 402 to a first end 401a of the second semi-circle portion 407.

A recessed opening 401b in the first end 401a of the second semi-circle portion 407 may receive a protruding portion 405b of the first end 405a of the first semi-circle portion 402. The pin 404b may pass through the first semi-circular portion 402 and the second semi-circular portion 407 where the recessed opening 401b and the protruding portion 405b create an overlap between the semicircular portions 402 and 407, such as through a hole in the first end 405a of the first semi-circle portion 402 and the first end 401a of the second semi-circular portion 407. The pin 404b may join the first semi-circular portion 402 to the second semi-circular portion 407, such as to form the hinged joint 404a.

A second end 406a of the first semi-circular portion 402 and a second end 408a of the second semi-circular portion 407 may be configured to interface with each other at a passive latch 403. The passive latch 403 may include a first shaped protruding portion 406b of the second end 406a and a first shaped receiving portion 408b of the second end 408a. The passive latch 403 may secure the first semi-circular portion 402 against the second semi-circular portion 407, thereby preventing rotation around the hinged joint. For example, the first shaped protruding portion 406b of the second end 406a of the first semi-circular portion 402 may be configured as a latching tab that may be inserted into the first shaped receiving portion 408b of the second end 408a of the second semi-circular portion 407 that may be configured as a latching recess.

When inserted into the latching recess, the latching tab may secure the first semi-circular portion 402 to the second semi-circular portion 407. In some embodiments, the catheter actuator collar 400 may be further configured to be retained in a joined relation by an enclosure, such as a drive linkage, as will be described in greater detail hereinafter. One or more tabs or projections 410 may be used to secure the catheter actuator collar 400 to the drive linkage.

When the second end 406 of the first semi-circular portion 402 is secured to the second end 408 of the second semi-circular portion 407, such as when secured within a drive linkage, the inner surface 409 of the first semi-circular portion 402 and the inner surface 411 of the second semi-circular portion 407 may securely grip a rotating catheter actuator 426 placed within the catheter actuator collar 400. In this manner, as the catheter actuator collar 400 is rotated, such as by the drive linkage, the actuator 426 may be rotated. In an embodiment, the catheter actuator collar 400 may be removed from the rotating catheter actuator 426 of a catheter handle 420 by removing the catheter actuator collar 400 from the drive linkage and separating the second end 406 of the first semi-circular portion 402 from the second end 408 of the second semi-circular portion 407 and rotating the first semi-circular portion 402 away from the second semi-circular portion 407 enabling the catheter actuator to be slid out or lifted out of the catheter actuator collar 400.

In some embodiments, the removable catheter actuator collar 402 may include the one or more projections 410 extending from an outer surface of the second semi-circular portion 410. While only one projection 410 is illustrated in FIG. 4A, additional projections 410 may extend from the outer surface of the second semi-circular portion 407 and/or the first semi-circular portion 402. While illustrated as a rectangular projection 410, the projection 410 may have any shape, such as square, triangular, rounded, etc.

The projection 410 may enable the removable catheter actuator collar 402 to couple with a drive linkage 434 of a component of a catheter positioning device, such as a sled 431. As shown in FIG. 4B, the projection 410 may fit into a recess 421 of a drive linkage 434, which may be driven by motors of the sled 431 to which the catheter handle 420 is coupled by way of a modular plate 430. By fitting within the recess 421, the catheter actuator collar 400 may be secured in a closed position such that the passive latch 403 provides an alignment function in addition to or in lieu of a latching function. The sled 431 may be configured with a first drive motor 442 that is coupled to a linkage 443 that may be used to drive the drive linkage 434. The catheter handle 420 may further be secured to the modular plate 430 by a movable clamp 432. The moveable clamp 432 may be driven along a linear axis by operation of a motor 444 and a linkage 445, which in some embodiments may be a worm gear or threaded rod that is inserted into a threaded opening of the linkage 445. Rotation of the motor 444 may rotate the worm gear or threaded rod, which in turn moves the linkage 445 and the movable clamp 432 back and forth along the linear axis.

In some embodiments, rotation of the drive linkage 434 including the recess 421 and the projection 410 disposed within the recess 421, may in turn rotate the catheter actuator collar 400. Rotation of the catheter actuator collar 400 may rotate the rotating actuator 426 of the catheter handle 420. The rotating drive linkage 434 may be a permanent part of the catheter positioning system, while the removable catheter actuator collar 400 may detach or decouple from drive linkage 434. As such, the removable catheter actuator collar 400 together with the catheter handle 420 held by the removable catheter actuator collar 400 may be removed from the drive linkage 434 and from the clamp 432 of the modular plate 430 and the catheter positioning system. The removable catheter actuator collar 400 may thereby be sterilized, resterilized, or disposed of.

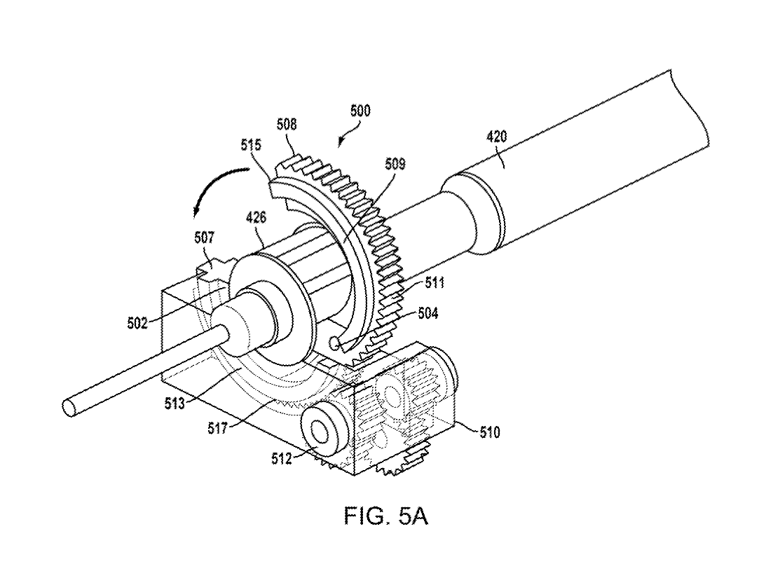

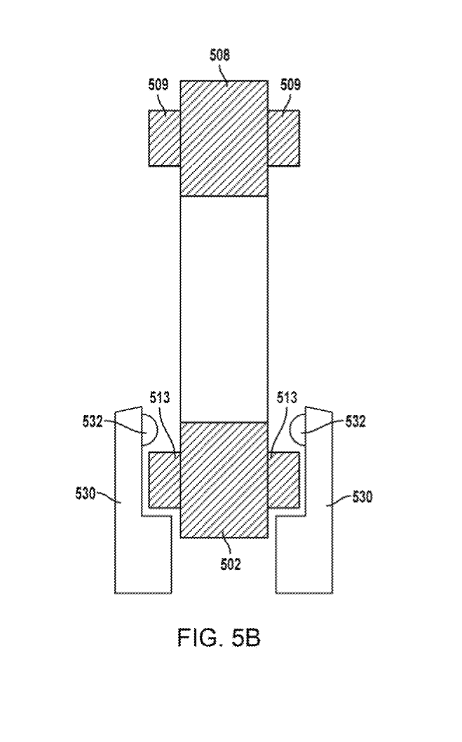

FIG. 5A illustrates another embodiment removable catheter actuator collar 500. The catheter actuator collar 500 may comprise a first semi-circular portion 508 and a second semi-circular portion 502. The first semi-circular portion 508 may be rotationally coupled to the second semi-circular portion 502 by a hinge 504. The hinge 504 may be configured to enable the first semi-circular portion 508 to rotate toward and/or away from the second semi-circular portion 502. Additionally, the first semi-circular portion 508 may be rotated about the hinge 504 toward the second semi-circular portion 502 such that an end 515 of the first semi-circular portion 508 may come into contact with an end 507 of the second semi-circular portion 502. The end 515 may contact the end 507 and the hinge 504 may hold the first semi-circular portion 508 and second semi-circular portion 502 in a closed position thereby forming a ring that may encircle the rotating catheter actuator 426. For example, in some embodiments, the hinge 504 may be configured with a spring mechanism (not shown) that urges the ends 515 and 507 into closed relation. In other embodiments, the hinge 504 may be a friction hinge, such that a certain degree of friction may be required to move the first and second semi-circular portions 508, 502 about the hinge 504. The friction may be related to a degree of force that is enough to hold the first semi-circular portion 508 and second semi-circular portion 502 together while overcoming any forces that may act to separate the two semi-circular portions.

The rotating catheter actuator 426 may be placed within the catheter actuator collar 500, which may securely grip the rotating catheter actuator 426. The first semi-circular portion 508 may include ridges 509 extending from a front and a back surface of the first semi-circular portion 508. The second semi-circular portion 502 may include ridges 513 extending from a front and a back surface of the second semi-circular portion 502. An outer surface of the first semi-circular portion 508 may include gear teeth 511 and an outer surface of the second semi-circular portion 502 may include gear teeth 517. The gear teeth 511 and 517 may interface with one or more linking gears of a drive mechanism 512 of a modular plate 510. The removable catheter actuator collar 500 and rotating actuator 426 may be rotated by driving the linking gears of the drive mechanism 512. In an embodiment, by “closing” the catheter actuator collar 500, such as by rotation of the first semi-circular portion 508 toward the second semi-circular portion 502 such that an end 515 of the first semi-circular portion 508 contacts an end 507 of the second semi-circular portion the gear teeth 511 and 517 and the ridges 509 and 513 may be aligned to form continuous ridges around the front and back sides of the catheter actuator collar 500 and a continuous set of gear teeth around the outer surface of the catheter actuator collar 500. In an embodiment, the catheter actuator collar 500 may be removed from an actuator 426 of a catheter handle 420 by separating the first semi-circular portion 508 from the second semi-circular portion 502 and rotating the first semi-circular portion 508 away from the second semi-circular portion 502 enabling the catheter actuator to be slid out or lifted out of the catheter actuator collar 500.

In an embodiment, the removable catheter actuator collar 500 may be sold or packaged with the modular plate 510 and may be provided already interfaced with the modular plate 510. For example, the gear teeth 517 of the removable catheter actuator collar 500 may come already meshed with one or more gears 512 of the modular plate 510 and/or the removable catheter actuator collar 500 may be snapped into the modular plate. In this manner, a physician may use a catheter manually and only attach the removable catheter actuator collar 500 to the catheter when the catheter is being coupled with the modular plate 510 of the catheter positioning system for use. A physician may subsequently detach and reattach the catheter and removable catheter actuator collar 500 to the modular plate 510.

In various embodiments, removable catheter actuator collars may couple and decouple with modular plate via various mechanisms. For example, FIG. 5B illustrates a cross sectional profile view of the removable catheter actuator collar 500 snapped into a clasp 530 of the modular plate 510. The ridges 509 and 513 may be snapped in and out of the clasp 530 by being urged past retaining structures 532 on either side of the clasp 530. The retaining structures 532 may take various forms, such as ridges or bumps, that keep the removable catheter actuator collar 500 coupled with the modular plate 510 as the linkage rotates but that allow the linkage to be removed by the physician or operator. The retaining structures 532 including the sides of the clasp 530 may flex to enable the removable catheter actuator collar 500 to be snapped into the clasp 530 and removed from the clasp 530. Additionally, the clasp 530 and retaining structures 532 may act as a guide enabling the catheter actuator collar 500 to rotate while supporting the catheter actuator collar 500.

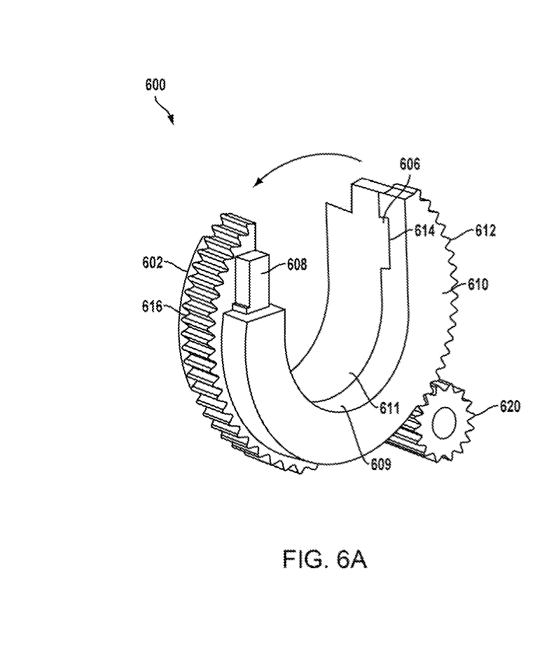

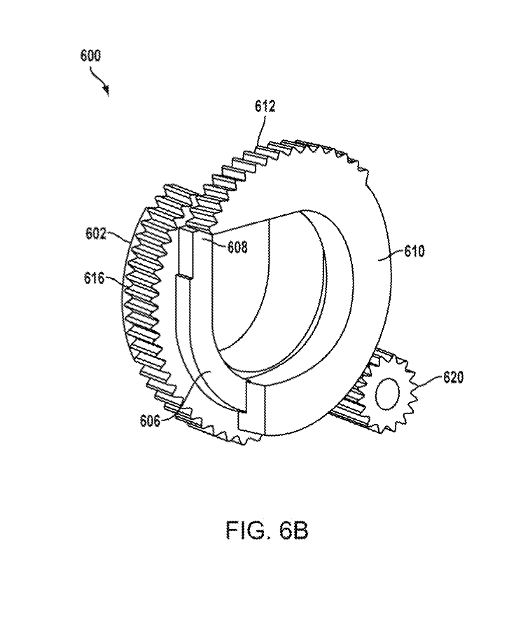

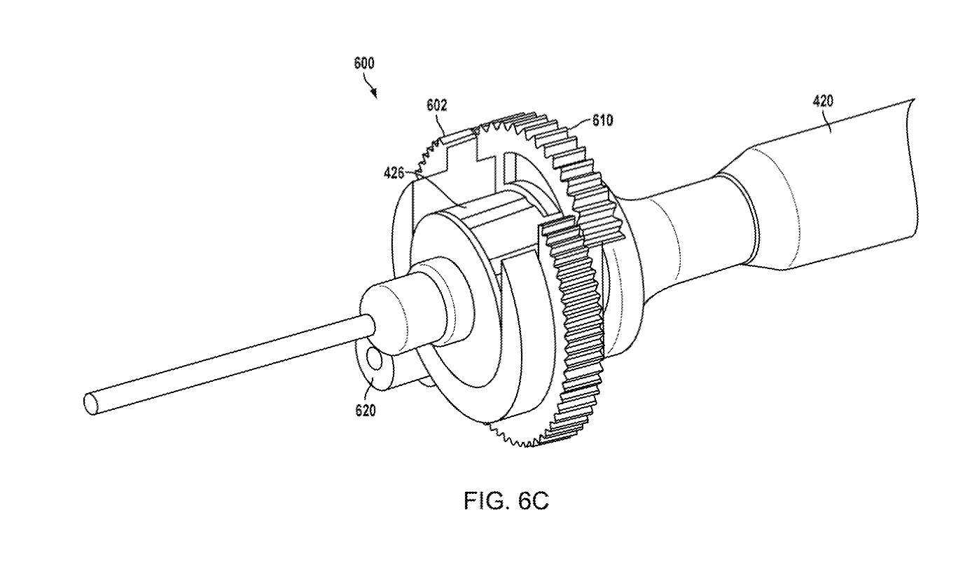

FIG. 6A illustrates another embodiment removable catheter actuator collar 600 comprising a first crescent portion 602 and second crescent portion 610 configured to rotate as indicated by the arrow to secure a catheter actuator inside the catheter actuator collar 600. FIG. 6A illustrates the removable catheter actuator collar 600 in an open position. The first crescent portion 602 may include gear teeth 616 extending radially from an outer surface and spaced along at least a portion of an outer surface of the first crescent portion 602. The second crescent portion 610 may include gear teeth 612 extending radially from an outer surface and spaced along at least a portion of an outer surface of the second crescent portion 610. In an embodiment, the gear teeth 616 may extend the full length of the outer surface of the first crescent portion 602 while the gear teeth 612 may extend less than the full length of the outer surface of the second crescent portion 610. The gear teeth 612 and 616 may interface with gear teeth of a linkage gear 620 of a catheter positioning system. The first crescent portion 602 and the second crescent portion 610 may be aligned such that the second crescent portion 610 is in contact with a face of the first crescent portion 602 and the second crescent portion 610 may rotate over the face of the first crescent portion 602 to establish a closed position. The first crescent portion 602 may include a ridge 606 that engages a recess or groove 614 in the second crescent portion 610, in which the ridge 606 may move along as the first crescent portion 602 rotates relative to the second crescent portion 610. The groove 614 of the second crescent portion 610 may be formed in the face of the second crescent portion that contacts the face of the first crescent portion 602. The groove 614 may be configured to align with the ridge 606 to allow the crescent portions 602, 610 to rotate relative to one another. In the open position, a recess 611 in the first crescent portion 602 may align with a recess 609 in the second crescent portion 610 to form an open recess that may receive a catheter actuator when open. The first crescent portion 602 may also include a projection 608 to hold the second crescent portion 610 in place to secure the removable catheter actuator collar 600 around a catheter actuator when closed.

FIG. 6B illustrates the removable catheter actuator collar 600 in a closed position. To achieve a closed position, the second crescent portion 610 may slide along the ridge 606 on the face of the first crescent portion 602. When the first crescent portion 602 and the second crescent portion 610 are slid into closed relation to form a ring, the orientation of the respective recess portions 611 and 609 may form an enclosed area in the center of the removable catheter actuator collar 600. When slid into the closed position, the surface of the recessed portion 611 and the surface of the recessed portion 609 may securely grip a rotating catheter actuator placed within the catheter actuator collar 600. In this manner, as the catheter actuator collar 600 is rotated, the actuator 426 of the catheter handle 420 may also be rotated. In an embodiment, the catheter actuator collar 600 may be removed from the actuator 426 of the catheter handle 420 by sliding the second crescent portion 609 back to the open position enabling the catheter actuator 426 to be lifted out of the catheter actuator collar 600.

FIG. 6C illustrates the removable catheter actuator collar 600 secured around the rotating actuator 426 of the catheter handle 420. The first crescent portion 602 and second crescent portion 610 may be rotated into the closed position to securely grip the rotating catheter actuator 426 placed within the catheter actuator collar 600. In the closed position, the combination of the gear teeth 612 and the gear teeth 616 may form a gear surface that positioned along the entire outer circumference of the removable catheter actuator collar 600. In this manner, rotation of the linkage gear 620, may rotate the removable catheter actuator collar 600 and thereby rotate the rotating actuator 426. In an embodiment, the linkage gear 620 may be a permanent part of a catheter positioning system. The removable catheter actuator collar 600 may detach or decouple from linkage gear 620 such that the removable catheter actuator collar 600 together with the catheter handle 420 and the rotating actuator 426 held by the removable catheter actuator collar 600 may be removed from the linkage gear 620 and the catheter positioning system.

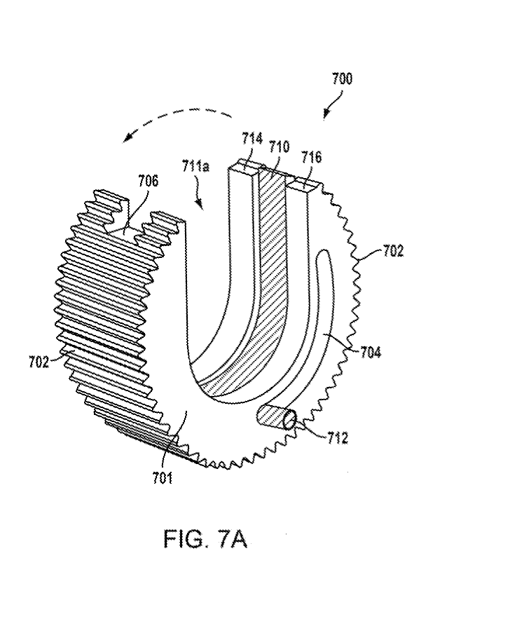

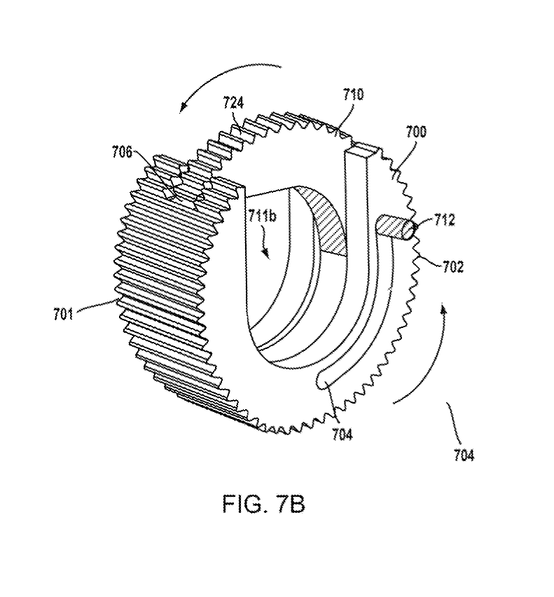

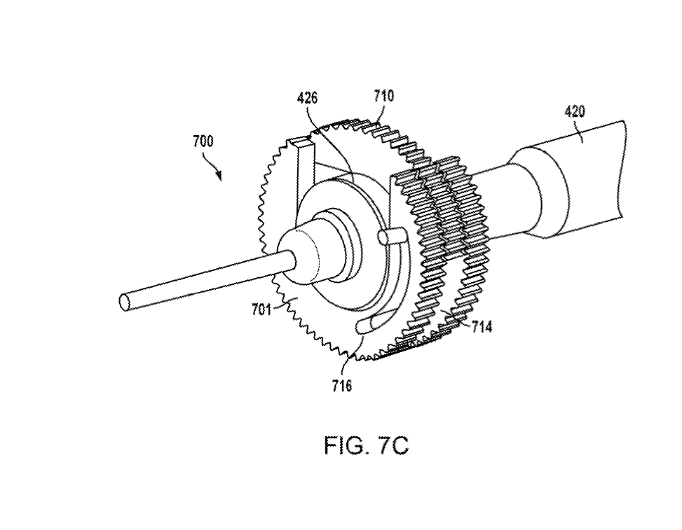

FIG. 7A illustrates another embodiment removable catheter actuator collar 700. The catheter actuator collar 700 is illustrated in FIG. 7A in an open position. The removable catheter actuator collar 700 may comprise a main body 701 having two upwardly extending arm portions 714 and 716. The two arm portions 714 and 716 may support a rotating portion 710 positioned between the two arm portions 714 and 716. In the open position, the rotating portion 710 may be aligned with the two arm portions 714 and 716 such that an opening 711 is formed in the removable catheter actuator collar 700. The opening 711 may be configured to accommodate a catheter actuator, such as the rotating actuator 426. In some embodiments, the gear teeth 702 may extend from the outer surface of the main body 701 and two arm portions 714 and 716. One or more of the arm portions 714 and/or 716 may be configured to include a curved slot 704 formed on the side or sides of the one or more of the arm portions 714 and 716. The curvature of the slot 704 may have a radius that corresponds generally to the circular radius of a curved portion of the main body 701 and the rotating portion 710. The slot 704 may be configured such that a projection 712 of the rotating portion 710 may protrude through the slot 704, which may function as a guide for the projection 712 for guiding the movement of the rotating portion 710. Movement of the projection 712 along the slot 704 may correspond to rotation of the rotating portion 710. In some embodiments, the projections 712 may be used to move the rotating portion 710. Rotational movement of the rotating portion 710 may transition the removable catheter actuator collar 700 from an open to a closed position. The main body 701 may include a recess 706 configured to receive at least a portion of the rotating portion 710 in the closed position.

FIG. 7B illustrates the removable catheter actuator collar 700 in a closed position. The projection 712 may slide along the slot 712 extending the rotating portion 710 out from between the arm portions 714 and 716. The rotation portion 710 may connect with the recessed portion 706 of the main body 701 forming an opening 711b enclosing an area in the center of the removable catheter actuator collar 700. The gear teeth 724 extending from an outer surface of the rotating portion 710 may align with the gear teeth 702 of the main body 701 and arm portions 714 and 716 thereby forming a continuous outer ring of gear teeth surrounding the opening 711b of the removable catheter actuator collar 700 in the closed position. When the rotating portion 710 is slid into the closed position, the inner surfaces of the main body 701, arm portions 714, 716, and rotating portion 710 may securely grip a rotating catheter actuator, such as the rotating actuator 426 illustrated in FIG. 4A, which may be placed within the catheter actuator collar 700. In this manner, as the catheter actuator collar 700 is rotated, the rotating actuator 426 may be rotated as well. In an embodiment, the catheter actuator collar 700 may be removed from the rotating actuator 426 of the catheter handle 420 by sliding the projection 712 within the slot 704 back to the open position enabling the rotating actuator 426 and the catheter handle 420 to be lifted out of the catheter actuator collar 700.

FIG. 7C illustrates the removable catheter actuator collar 700 in a closed position and secured around the rotating actuator 426 of a catheter handle 420. In some embodiments, the projection 712 may slide along the slot 712 extending the rotating portion 710 from between the arm portions 714 and 716 to connect with the recessed portion 706 of the main body 701 as discussed above in order to securely grip the rotating catheter actuator 426 placed within the catheter actuator collar 700. In an embodiment, the removable catheter actuator collar 700 may detach or decouple from a linkage gear used to drive the removable catheter actuator collar 700 such that the removable catheter actuator collar 700 together with the catheter held by the removable catheter actuator collar 700 may be removed from the linkage gear and the catheter positioning system. The removable catheter actuator collar 700 may be removed to be sterilized, re-sterilized, or for disposal.

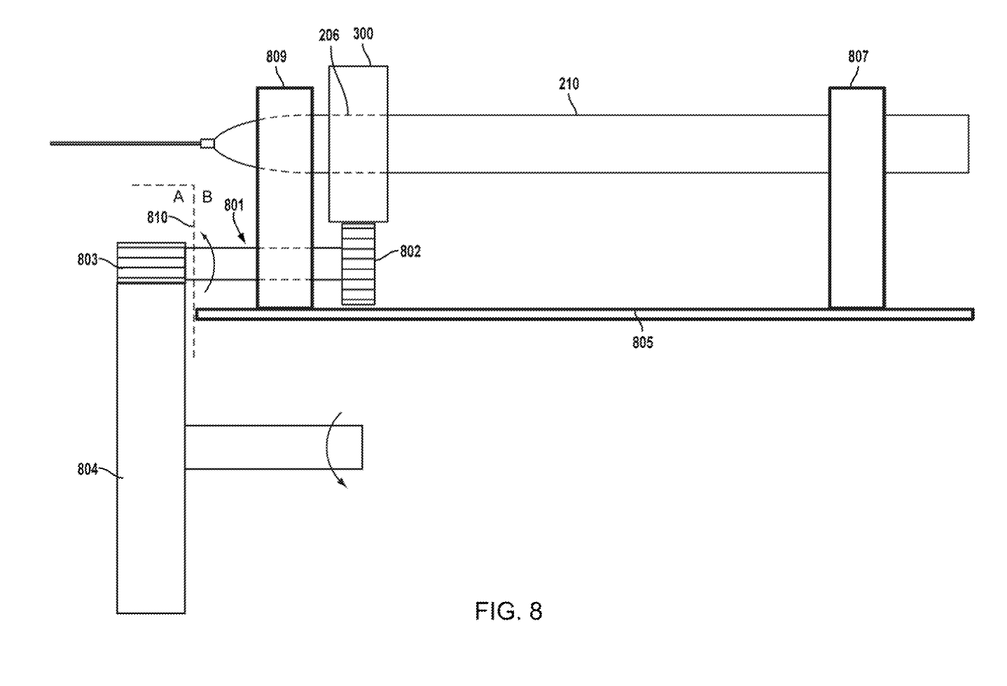

In the various embodiments, the catheter contacting components of the removable catheter actuator collars may be sterile components, either sterilizable or disposable, to avoid introducing contaminants into the body of a patient. In the various embodiments, sterility may be maintained using a variety of approaches individually or in combination, including barriers, multi-stage interfaces, distance/separation, etc. For example, as illustrated in FIG. 8, sterility may be maintained by using a sterile intermediate shaft 801 as part of a two stage interface to actuate the rotating actuator of the catheter handle 210. A two stage interface may be an interface having a non-sterile side A and a sterile side B relative to a sterility boundary 810. A first stage engages with a drive mechanism on the side A of the sterility boundary 810 and may not remain sterile. A second stage engages with the actuator on the side B of the sterility boundary 810 and may remain sterile. While the sterility boundary 810 is shown as separating the A and B sides, the boundary may be positioned differently. For example, the sterility boundary may encompass more catheter components on the sterile B side. FIG. 8 illustrates a removable catheter actuator collar 300 affixed around a rotating catheter actuator 206 of a catheter handle 210, such as described above with reference to FIG. 3A. The two stage interface illustrated in FIG. 8 may comprise the intermediate shaft 801 as a first stage and the removable catheter actuator collar 300 as a second stage. The catheter handle 210 may be supported in a sterile docking plate 805 attached to a sled member. The sled member may include a rotating linkage 804, which may be driven by a motor (not shown). The rotating linkage 804 may not be sterile. The docking plate 805 may include two catheter mounts 807 and 809 to support the catheter handle 210. The intermediate shaft 801 may be supported in the catheter mount 809 such that the intermediate shaft 801 may rotate and a first set of gear teeth 803 may interface with the rotating linkage 804 when the docking plate 805 is placed on the sled member. A second set of gear teeth 802 may interface with the removable catheter actuator collar 300 when the catheter handle 210 is supported in the catheter mounts 807 and 809 of the docking plate 805. The rotation of the rotating linkage 804 may rotate the intermediate shaft 801, which may in turn rotate the removable catheter actuator collar 300 to rotate the rotating actuator of the catheter handle 210. The separation of the removable catheter actuator collar 300 from the rotating linkage 804 may enable a user to remove the catheter handle 210 from the docking plate 805 without breaking sterility of the catheter or removable catheter actuator collar 300.

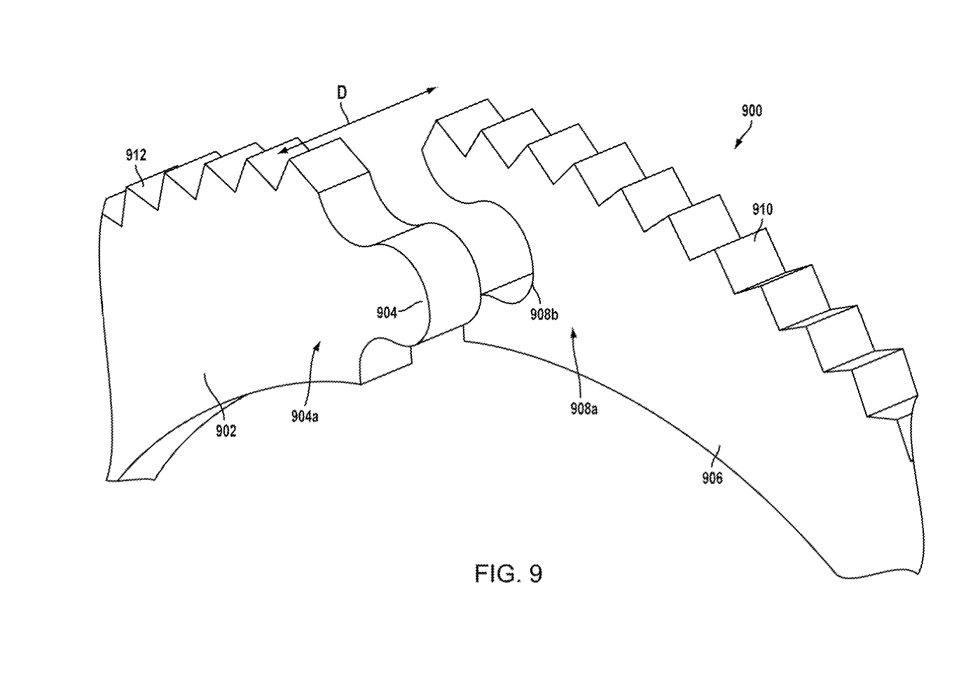

FIG. 9 illustrates a portion of an alternate embodiment removable catheter actuator collar 900. The catheter actuator collar 900 may comprise a first semi-circular portion 902 and a second semi-circular portion 906. The first semi-circular portion 902 may be rotationally coupled to the second semi-circular portion 906 by a hinge as described herein. The hinge may be configured to enable the first semi-circular portion 902 to rotate toward and/or away from the second semi-circular portion 906. Additionally, the hinge may be laterally flexible so as to be configured to enable the first semi-circular portion 902 and the second semi-circular portion 906 to be deflected toward and/or away from each other a distance D in a plane perpendicular to the plane of rotation of the first semi-circular portion 902 and second semi-circular portion 906 around the hinge. Such deflection may allow the first and second semi-circular portions 902, 906 to be joined or separated.

An end 904a of the first semi-circular portion 902 may be configured to form a protrusion 904b and an end 908a of the second semi-circular portion 906 may be configured to form a recess 908b shaped so as to receive the protrusion 904b. When the first semi-circular portion 902 is rotated and deflected toward the second semi-circular portion 906 such that the protrusion 904b of the end 904a of the first semi-circular portion 902 fits within the recess 908b of the end 908a of the second semi-circular portion 906, the protrusion 904b and the recess 908b may form a snap for holding the first semi-circular portion 902 and second semi-circular portion 906 in a closed position. The closed configuration of the protrusion 904b and the recess 908b may thereby form a ring that may encircle and securely grip a rotating catheter actuator, such as the actuator 426, placed in the catheter actuator collar 900. An outer surface of the first semi-circular portion 902 may include gear teeth 912 and an outer surface of the second semi-circular portion 906 may include gear teeth 910. The gear teeth 912 and 910 may interface with one or more linking gears of a modular plate. The removable catheter actuator collar 900 and rotating actuator of a catheter may be rotated by driving the linking gears. In this manner, as the catheter actuator collar 900 is rotated, the actuator of the catheter may be rotated as well. In an embodiment, rotation of the first semi-circular portion 902 toward the second semi-circular portion 906, such that the protrusion 904b of the end 904a of the first semi-circular portion 902 snaps into the recess 908b of the end 908a of the second semi-circular portion 906, may align the gear teeth 912 and 910 to form a continuous set of gear teeth around the outer surface of the catheter actuator collar 900. In an embodiment, the catheter actuator collar 900 may be removed from an actuator of a catheter handle by separating the first semi-circular portion 902 from the second semi-circular portion 906 by deflecting and rotating the first semi-circular portion 902 away from the second semi-circular portion 906 enabling the catheter actuator to be slid out or lifted out of the catheter actuator collar 900. In this manner, the catheter actuator collar 900 may be easily unsnapped, for example with a swipe of a single finger, to allow the catheter actuator to be slid or lifted out of the catheter actuator collar 900.

Those skilled in the art will recognize that the methods and systems of the present embodiments have many applications, may be implemented in many manners and, as such, are not to be limited by the preceding exemplary embodiments and examples. Additionally, the functionality of the components of the preceding embodiments may be implemented in different manners. Further, it is to be understood that the steps in the embodiments may be performed in any suitable order, combined into fewer steps or divided into more steps. Thus, the scope of the present invention covers conventionally known and future developed variations and modifications to the system components described herein, as would be understood by those skilled in the art.

What is claimed is:

1. A removable catheter actuator collar, comprising:
an inner ring having one or more protrusions extending from an inner circumference of the inner ring into a central opening of the inner ring; and
an outer ring surrounding the inner ring, an inner surface of the outer ring coupled to an outer surface of the inner ring such that rotation of the outer ring rotates the inner ring,
wherein the one or more protrusions are configured to grip a catheter actuator, and
wherein the outer ring is configured to interface with an actuator of a remote catheter positioning system.

2. The removable catheter actuator collar of claim 1, wherein the one or more protrusions are at least partially flexible protrusions and configured to deform when in contact with the catheter actuator.

3. The removable catheter actuator collar of claim 2, wherein the outer ring is configured to restrict an expansion of a circumference of the inner ring such that the one or more at least partially flexible protrusions apply a force against the catheter actuator when deformed.

4. The removable catheter actuator collar of claim 1, wherein the one or more protrusions comprise a curved contact surface for contacting the catheter actuator.

5. The removable catheter actuator collar of claim 1, wherein the inner ring is comprised of a different material than the outer ring.

6. The removable catheter actuator collar of claim 5, wherein the one or more protrusions include four protrusions.

7. A removable catheter actuator collar, comprising:
a main body section;
a clamp rotationally coupled to the main body section; and
an adjustable clamping section coupled to the clamp, the adjustable clamping section comprising a clamping arm in adjustable contact with the clamp,
wherein rotation of the clamp to a first position causes the clamping arm to move to reduce an inner circumference of the adjustable clamping section to grip a catheter actuator;
wherein rotation of the claim to a second position causes the clamping arm to move to increase the inner circumference of the adjustable clamping section to release a grip on the catheter actuator, and
wherein an outer portion of the main body section is configured to interface with an actuator of a remote catheter positioning system.

8. The removable catheter actuator collar of claim 7, wherein the main body section comprises gear teeth extending from an outer circumference of the main body section.

9. The removable catheter actuator collar of claim 8, wherein the clamp is rotationally coupled to the main body section by a pin and wherein the clamp rotates about the pin to the first and the second positions.

10. The removable catheter actuator collar of claim 7, wherein an outer portion of the main body section is configured to interface with an actuator of a remote catheter positioning system through a multi-stage interface.

11. The removable catheter actuator collar of claim 10, wherein the multi-stage interface comprises a two stage interface configured to enable the catheter actuator that is gripped by the clamp and the removable catheter actuator collar to be removed from the actuator of the remote catheter positioning system while maintaining a sterility of the removable catheter actuator collar.

12. A removable catheter actuator collar, comprising:
a first semi-circular portion having a first inner surface, a first end, and a second end; and
a second semi-circular portion having a second inner surface, a third end, and a fourth end, the third end rotationally coupled to the first end,
wherein the second end and the fourth end are configured to secure together to form a ring such that the first inner surface and the second inner surface are configured to grip a catheter actuator, and
wherein an outer portion of the ring of the removable catheter actuator collar is configured to interface with an actuator of a remote catheter positioning system.

13. The removable catheter actuator collar of claim 12, wherein at least one of the first semi-circular portion and the second semi-circular portion includes a projection extending from the outer portion of the second semi-circular portion, the projection configured to be secured within a recess of a linkage of a catheter drive system.

14. The removable catheter actuator collar of claim 13, wherein the third end is rotationally coupled to the first end by a pin and first semi-circular portion and the second semi-circular portion rotate about the pin.

15. A removable catheter actuator collar, comprising:
a first semi-circular portion comprising:
a first set of gear teeth extending from around a first outer surface of the first semi-circular portion;
a first semi-circular ridge extending from a first front surface of the first semi-circular portion; and
a second semi-circular ridge extending from a first back surface of the first semi-circular portion;
a second semi-circular portion comprising:
a second set of gear teeth extending from a second outer surface of the second semi-circular portion;
a third semi-circular ridge extending from a second front surface of the second semi-circular portion; and
a fourth semi-circular ridge extending from a second back surface of the second semi-circular portion; and
a hinge rotationally coupling the first semi-circular portion to the second semi-circular portion,
wherein the hinge is configured to secure the first semi-circular portion and the second semi-circular portion together to grip a catheter actuator and to align the first semi-circular ridge and the second semi-circular ridge with the third semi-circular ridge and the fourth semi-circular ridge and to align the first set of gear teeth with the second set of gear teeth, and
wherein the first and second set of gear teeth are configured to interface with an actuator of a remote catheter positioning system.

16. The removable actuator collar of claim 15, wherein the hinge comprises a spring hinge that holds the first semi-circular portion and the second semi-circular portion together in closed relation.

17. The removable actuator collar of claim 15, wherein the first semi-circular ridge, the second semi-circular ridge, the third semi-circular ridge and the fourth semi-circular ridge secure the removable actuator collar within a modular plate and secure the first and second sets of gear teeth of the removable actuator collar against a drive mechanism of the actuator of the remote catheter positioning system.

18. The removable actuator collar of claim 17, wherein the first semi-circular ridge, the second semi-circular ridge, the third semi-circular ridge and the fourth semi-circular ridge are secured within the modular plate by a flexible clasp coupled to the modular plate.

19. A removable catheter actuator collar, comprising:
a first crescent portion having a first set of gear teeth extending from along a first outer surface; and
a second crescent portion having second set of gear teeth extending from along a second outer surface,
wherein the second crescent portion is configured to rotate across a face of the first crescent portion to form a outer ring comprising the first and second set of gear teeth and an inner ring to grip a catheter actuator and align a portion of the first set of gear teeth with the second set of gear teeth, and
wherein the outer ring including the first and second set of gear teeth is configured to interface with an actuator of a remote catheter positioning system.

20. The removable catheter actuator collar of claim 19, wherein the first crescent portion includes a ridge extending from the face of the first crescent portion and the second crescent portion includes a groove configured to align with the ridge and guide a rotation of the second crescent portion across the face of the first crescent portion.

21. The removable catheter actuator collar of claim 19, wherein the second set of gear teeth extend from along the second outer surface over a length less than the entire length of the second outer surface.

22. A removable catheter actuator collar, comprising:
a main body;
a first arm portion extending from the main body;
a second arm portion extending from the main body; and
a rotating arm portion movably positioned between the first arm portion and the second arm portion,
wherein the rotating arm portion is configured to rotate from between the first arm portion and the second arm portion to grip a catheter actuator, and
wherein an outer portion of the removable catheter actuator collar is configured to interface with an actuator of a remote catheter positioning system.

23. The removable catheter actuator collar of claim 22, wherein the outer portion comprises an outer surface of a portion of the main body, a portion of the first arm portion, a portion of the second arm portion, and portion of the rotating arm portion.

24. The removable catheter actuator collar of claim 22, wherein an outer surface of the main body, an outer surface of the first arm portion, an outer surface of the second arm portion, and an outer surface of the rotating arm portion include gear teeth extending from along the respective outer surfaces and wherein when the rotating arm portion is rotated from between the first arm portion and the second arm portion to grip the catheter actuator the gear teeth surround the outside of the removable catheter actuator collar.

25. The removable catheter actuator collar of claim 24, wherein at least one of the first arm portion and the second arm portion includes a slot and wherein the rotating arm portion includes a projection extending through the slot, and wherein sliding the projection along the slot rotates the rotating portion from between the first arm portion and the second arm portion to grip the catheter actuator.

26. A removable catheter actuator collar, comprising:
a first semi-circular portion having a first set of gear teeth extending from along a first outer surface, and a first end of the first semi-circular portion configured to form a protrusion;
a second semi-circular portion having a second set of gear teeth extending from along a second outer surface, and a second end of the second semi-circular portion configured to form a recess; and
a hinge rotationally coupling the first semi-circular portion to the second semi-circular portion,
wherein the protrusion and the recess are configured to snap together to secure the first semi-circular portion and the second semi-circular portion together to grip a catheter actuator and align the first set of gear teeth with the second set of gear teeth, and
wherein the first and second set of gear teeth are configured to interface with an actuator of a remote catheter positioning system.
